(19) United States
(12) Patent Application Publication
(10) Pub. No.: US 2021/0369499 A1
(43) Pub. Date: Dec. 2, 2021

(54) DEVICE FOR OPHTHALMOLOGIC SURGERY

(71) Applicant: José LAMARCA MATEU, Barcelona (ES)

(72) Inventor: José LAMARCA MATEU, Barcelona (ES)

(21) Appl. No.: 16/982,948

(22) PCT Filed: Apr. 10, 2019
(86) PCT No.: PCT/ES2019/070251

§ 371 Date: Sep. 21, 2020

(30) Foreign Application Priority Data
Apr. 10, 2018 (ES) U201830501

(51) Int. Cl. A61F 9/007; A61B 17/12

(52) U.S. Cl. CPC A61F 9/007; A61B 2017/00876; A61B 2017/12004; A61B 17/12009

(57) ABSTRACT

A device (1) for eye surgery includes a metallic ring (2) for suturing to the sclera of a patient's eye. A locking element (3) can be coupled to the metallic ring (2). The locking element (3) includes a lens (31) and a coupling ring (32) fixed to the lens (31) along the entire circumference of the inner edge of the lens (31). The coupling ring (32) is configured for coupling to the metallic ring (2).

### OBJECT OF THE INVENTION

This invention belongs to the field of ophthalmology.

The object of the present invention is a device designed for:
1. Stopping an expulsive ocular haemorrhage immediately during ocular surgery.
2. Improving intraoperative visualisation of the inside of the eyeball during keratoplasty surgeries.
3. Facilitating posterior segment surgery (including but not limited to laser surgery) during combined keratoplasty and posterior segment surgeries.
4. Performing experimental intraocular surgery especially when the cornea has opacities.

In addition, it is a system that allows the use of different types of lenses in the context of eye surgery.

### BACKGROUND OF THE INVENTION

Expulsive ocular haemorrhage (EOH) is one of the most serious complications in the context of ocular surgery. The predisposing factors of EOH are arteriosclerosis and/or hypertension in elderly patients, increased intraocular pressure, increased extraocular pressure, decreased scleral stiffness and severe myopia. During surgery there are other predisposing factors such as sudden increase in blood pressure, cough, loss of vitreous humour, as well as any pressure on the eyeball. EOH may occur in any type of intraocular surgery, although there is a special predisposition in surgeries wherein the intraocular content is exposed, as it happens, among others, in penetrating keratoplasty surgery. The appearance of EOH is usually unexpected and, due to this, the surgeon is not usually prepared to treat it efficiently immediately.

The main goal of treating EOH is to stop the bleeding as soon as possible. Currently, this is done either with a rapid suture in those cases wherein the only communication between the inside and outside of the globe is a solution of continuity, or in cases of corneal transplant surgery by applying the finger on the surgical wound in order to plug it or using a blocking lens. Once the situation is stabilised, posterior sclerotomies are performed that allow the drainage of blood from the supracoroid haemorrhage, with or without the aid of intraocular surgery to replace structures such as the retina.

However, at present there is no sufficiently rapid and effective solution as would be desirable.

In the process of EOH treatment the origin and possible related complications such as the detachment of isolated choroids or associated with retinal detachment must be verified. A correct visualisation is essential for a correct therapeutic decision.

In this sense, not only in cases of EOH during penetrating keratoplasty but also in those of combined surgery involving a complete corneal and retina transplant, a correct visualisation is required that until now has been made through the use of temporary keratoprosthetics anchored to the ocular wall through sutures. In the context of an EOH the suture is difficult because the displaced intraocular content hinders the process. In addition, in the surgical process, certain lenses are necessary that require the collaboration of an assistant in order to obtain a correct visualisation.

Finally, part of the experimental ophthalmologic surgery is developed with human eyeballs as well as those from other species. Generally, the main problem that is usually found is the opacity of corneal media caused by the passage of hours due to the interruption of the metabolism. In this sense, there are no alternatives to temporary keratoprosthetics.

### DESCRIPTION OF THE INVENTION

The present invention solves the above problems by means of a novel device particularly designed to stop an EOH almost immediately. The particular design of this device also allows the performance of combined surgery of penetrating keratoplasty and posterior segment of the eyeball, as well as surgery of the experimental posterior segment in globes with media opacity.

The present invention is directed to a device for ophthalmological surgery comprising mainly two components: a metallic ring; and a locking element attachable to said metallic ring. Each of these components is described in more detail below.

A) Metallic Ring

This is a structural ring suitable to be sutured to the sclera of a patient's eye. Rings similar to this one, such as the Flieringa ring, are commonly used in this field to avoid the collapsing of the anterior segment when certain interventions are performed, such as keratoplasty combined with cataract extraction or extracapsular extraction in an eye with very high level of myopia. The main function of the metallic ring is purely structural and so the material of which it is made is primarily a biocompatible metal commonly used in the field of ophthalmology, such as steel, nitinol or titanium. However, the ring may be covered or contain a series of modules to facilitate its attachment to the locking element. The geometry of these modules makes it possible for the ring to be adapted to the surface of the eyeball, as well as to fix it to the locking element by means of sealing mechanisms described in the following sections. The number of modules that the ring can have may be from 0 to 50 and the material of which the modules are made may be any of the biocompatible materials used in the field of ophthalmology such as steel, titanium or polymeric base: polyethylene (PE), polyethylene terephthalate (PEFT), polypropylene (PP), polycaprolactone (PEEK), polyamide (PA), polytetrafluoroethylene (PTFE), polyurethane (PU-TPU), polyarylketone (PAA), polystyrene (PS), polyvinyl chloride (PVC), acrylonitrile butadiene styrene (ABS), poly lactic acid (PLA) or similar either in terms of molecular weight or the mixture of various materials.

The diameter of the metallic ring is similar to that of similar rings currently used in the context of ophthalmology, such as the Flieringa ring. For example, the metallic ring of the present invention may have a diameter between 11 mm and 24 mm.

As for the shape of its cross section, the metallic ring of the invention may have a cross section of different shapes as long as they allow it to be attached to the locking element, as will be explained below in this document. For example, the metallic ring may have a circular cross section, in which case it would be similar to the Flieringa ring. Alternatively, the metallic ring may have a parallelepipedic cross section, e.g. rectangular, square, or similar. The shape of the cross section of the modules attached to the metallic ring may coincide with the shape of the cross section of the metallic ring and may have a circular or parallelepipedal shape.

Optionally, the metallic ring may contain one or more protrusions (“tabs”) of the same materials used in the metallic ring or in the modules to be used as fasteners and facilitate the separation of the closure element when required during the surgical process.

b) Locking Element

This is a locking element that can be fitted to the metallic ring and which comprises two different parts: a lens and a coupling ring.

b1) Coupling ring (magnetic, mechanical or sealed connection)
The coupling ring has the function of ensuring the tightness/fixation of the system by joining the metallic ring to the lens. The coupling ring is attached to the lens along the entire circumference of the inner or outer edge of the lens. In addition, the coupling ring can be made of any of the biocompatible materials used in the field of ophthalmology such as steel, titanium, nitinol or polymeric base: polyethylene (PE), polyethylene terephthalate (PEFT), polypropylene (PP), polycaprolactone (PEEK), polyamide (PA), polytetrafluoroethylene (PTFE), polyurethane (PU - TPU), polyarylketone (PAA), polystyrene (PS), polyvinyl chloride (PVC), acrylonitrile butadiene styrene (ABS), poly lactic acid (PLA) or similar either in terms of molecular weight or the mixture of various materials. In addition, it can be a magnet with magnetic properties e.g. of neodymium, an electromagnet, or it can be covered or contain a series of modules to facilitate its coupling to the metallic ring. The geometry of these modules allows the coupling ring to be adapted and/or fixed to the metallic ring by means of sealing mechanisms described in the following sections. The number of modules that the coupling ring can have may be from 0 to 50 and the material of which the modules are made may be any of the biocompatible materials used in the field of ophthalmology such as steel, titanium or polymeric base: polyethylene (PE), polyethylene terephthalate (PEFT), polypropylene (PP), polycaprolactone (PEEK), polyamide (PA), polytetrafluoroethylene (PTFE), polyurethane (PU-TPU), polyarylketone (PAA), polystyrene (PS), polyvinyl chloride (PVC), acrylonitrile butadiene styrene (ABS), poly lactic acid (PLA) or similar either in terms of molecular weight or the mixture of various materials.
In addition, the coupling ring has the particular characteristic of being configured for coupling to the metallic ring by means of one or more of the connections described below:
   Magnetic connection: The magnetic connection consists of the electromagnetic force closing of the coupling ring with the metallic ring. For this purpose, both in the modules present in the coupling ring or in the metallic ring itself, a magnetic material and a ferromagnetic material must be used to guarantee the connection. For this purpose, the modules of the coupling ring and/or the metallic ring may be and/or contain one or more magnetic elements, preferably neodymium magnets or electromagnets.
   Mechanical connection: The mechanical connection consists of the mechanical closing of the coupling ring with the metallic ring. For this purpose, the coupling ring and/or its modules shall have a grooved and protruding or threaded mechanism which allows the metallic ring to be fixed and/or sealed with the coupling ring.
   Sealed connection: The sealed connection consists of the closing by means of an elastomeric material of the coupling ring with the metallic ring. The coupling ring is made of an elastomer material, preferably medical silicone or polyurethane.
The material of which the coupling ring is composed depends on the mechanism used to ensure fixation and/or closure and may be and/or contain any of the biocompatible materials used in the field of ophthalmology such as steel, titanium or polymeric base: polyethylene (PE), polyethylene terephthalate (PEFT), polypropylene (PP), polycaprolactone (PEEK), polyamide (PA), polytetrafluoroethylene (PTFE), polyurethane (PU - TPU), polyarylketone (PAA), polystyrene (PS), polyvinyl chloride (PVC), acrylonitrile butadiene styrene (ABS), poly lactic acid (PLA) or similar either in terms of molecular weight or the mixture of various materials. In the case of magnetic connection, the coupling ring may be or contain a magnetic or ferromagnetic material to ensure its closure, while for the sealed connection the coupling ring material shall be of elastomeric origin in the field of ophthalmology, preferably medical grade silicone or polyurethane.

Thanks to this configuration, as mentioned above, it is possible to use the metallic ring of the invention to perform functions similar to those of the Flieringa ring and, in the event of an EOH, the surgeon can quickly attach the locking element to the metallic ring by means of the coupling ring. Thanks to the lens attached to the coupling ring, bleeding is blocked much more quickly than with solutions currently in use. The diameter of the locking element can be between 12 mm and 18 mm.
In the device of the present invention, the coupling between the coupling ring and the metallic ring can be carried out in different ways.

In a preferred embodiment of the invention, the coupling ring has magnetic properties for the coupling of the metallic ring. That is, the coupling ring is shaped like a magnet in such a way that it attracts the metallic ring. This configuration is advantageous because it allows an almost immediate coupling of the locking element to the metallic ring. In principle, both the coupling ring and the metallic ring can have any cross section shape as long as it allows the described magnetic coupling. However, in a particularly preferred embodiment of the invention, the coupling ring has a cross section with a cavity whose shape is complementary to the shape of the cross section of the metallic ring. In other words, the coupling ring has a channel which runs along its entire circumference on one side and which has a cross section complementary to the cross section of the metallic ring. In this way, during the magnetic coupling described, the metallic ring would be inserted tightly into the cavity, thus being enclosed in its interior to prevent a possible decoupling. This configuration is advantageous because it increases the rigidity of the coupling between the locking element and the metallic ring.
Two examples are described below wherein the cross sectional shape of the metallic ring is complementary to the cross sectional cavity shape of the coupling ring. In a first example, the cavity of the coupling ring may be shaped like a parallelepiped (it could be rectangular, square, etc.), in which case the metallic ring would have a correspondingly parallelepipedic cross section (correspondingly, it could also be rectangular, square, etc.). In a second example, the cavity of the coupling ring may be semicircular, in which case the metallic ring would have a circular cross section. In this second example, the metallic ring would be very similar to a Flieringa ring.

In alternative invention embodiments, the coupling between the coupling ring and the metallic ring can be performed in other ways different from the magnetic coupling. For example, it may be a threaded coupling, in which case the coupling ring and the metallic ring include additional threads that enable them to be coupled. Alternatively, the device of the invention may in addition comprise fixing clips for the coupling between the coupling ring and the metallic ring. In general, any coupling system that allows a quick coupling between the two elements could be used.
In other alternative embodiments of the invention, the coupling ring may contain fixation (or anchorage) elements to be able to adapt in the same coupling ring different types of lenses for posterior segment surgery and for combined surgery of penetrating and posterior segment keratoplasty. These fixation elements can have different diameters and/or heights so that they can be optimally adapted to the different types of lenses available for surgery.

b2) Lens (1—lens for EOH; 2—Lens for combined surgery of penetrating and posterior segment keratoplasty; and 3—Lens for experimental posterior segment surgery)
The lens can in principle be of any shape, type and material as long as it blocks the bleeding when fixed to the metallic ring through the coupling ring.
In a preferred embodiment of the invention, the lens is a transparent lens made of plastic, for example of polymethylmethacrylate. In an alternative preferred embodiment, the lens is made of a material compatible with paracentesis, such as silicone.
In addition, the lens has a range of central curvature in its optical zone for correct visualisation and with the possibility of adapting lenses in the context of surgery combined with the posterior segment of the eyeball, being just as useful as the temporary keratoprosthetics currently used, with the advantage that the time of application is much faster.
Thus, given that the normal cornea has about 44 diopters (7.67 mm and 25.8), and bearing in mind that on the other hand, the lens design must allow for the adaptation of surgical lenses that assist in the visualisation of the posterior segment (curvature around 20° in the optical zone), the lens is similar to a rigid corneoscleral lens with a central curvature in its optical zone, transition zone and anchorage zone to the coupling ring.

The device of the invention adequately solves the problems described above, as it makes it possible to deal with an EOH in an almost immediate manner. Below is a brief description of how to use this device.

Currently, for EOHs in combined posterior segment eye surgery and keratoplasty, temporary keratoprosthetics (Eckardt or Landers) are used. In order to be able to place them, the picture must have been previously stabilised, which is quite a difficult question considering that although we can act with systemic drugs, we cannot act in a topical way. Keratoprosthetics should be placed using sutures and high molecular weight viscoelastics should be used to counteract bleeding. This process is slow and laborious, which can lead to loss of intraocular content as the closure is not immediate. Once the keratoprosthetics are in place, it is possible to act internally on the globe and externally with drainage sclerotomies.

When the device is used according to the invention, it is based on a situation in which the metal ring is sutured to the sclera. The use of metallic rings of this type sutured to sclera in penetrating keratoplasty surgeries, such as the Flieringa ring, is well known. The ring helps maintain the architecture of the eyeball when the inner contents are exposed by removing the injured cornea. For many years it has been used to counteract the hypotonia caused by the surgical procedure and thus decrease the likelihood of EOH. If an EOH occurs in this situation, it can be stopped almost immediately by coupling the locking element to the metallic ring. To do this, the coupling ring simply needs to be attached to the metallic ring by means of the magnetic, mechanical or sealed connection in question. The lens serves as a barrier to prevent bleeding.

This new device has a number of advantages over current surgical practices:
Before the sudden appearance of a EOH, the device of the invention allows immediate action to be taken for the rapid recovery of the hermeticity of the eyeball, avoiding further damage that could be irreparable. Current treatments require more time, thus increasing the risk of loss of intraocular content.
The device of the invention allows EOH to be treated in a simpler, cleaner and controlled way, without sutures, the use of viscoelastic, or temporary keratoprosthetics.
The device of the invention replaces the temporary keratoprosthetics, allowing the adaptation of surgical lenses that assist the visualisation of the posterior segment.
The device of the invention, in the version of it that allows paracentesis (e.g. silicone lens), allows surgery through the lens.
The device of the invention is useful in experimental surgery both in terms of experimental animals and with eyes that have been donated. This device could be especially useful for training novice surgeons who want to perform intraocular surgery on human globes, since corneal edema is often observed, making it impossible to perform certain surgeries. In this case the opaque cornea would be replaced by the device of invention.

### BRIEF DESCRIPTION OF THE FIGURES

FIGS. 1a and 1b respectively show a top view and a cross section of a first example of the metallic ring according to the present invention.

FIGS. 2a and 2b respectively show a top view and a cross section of a first example of the locking element according to the present invention.

FIGS. 3a and 3b respectively show a view of the first example of invention device before the coupling of the locking element to the metallic ring and after the coupling of the locking element to the metallic ring.

FIGS. 4a and 4b respectively show a top view and a cross section of a second example of the metallic ring according to the present invention.

FIGS. 5a and 5b respectively show a top view and a cross section of a second example of the locking element according to the present invention.

FIGS. 6a and 6b respectively show a view of the second example of invention device before the coupling of the locking element to the metallic ring and after the coupling of the locking element to the metallic ring.

FIGS. 7 and 8 show an example of a metallic ring of the invention. In the case of

FIG. 8, a perspective view of half a metallic ring is shown.

FIG. 9 shows an example of a device of the invention with a metallic ring and a locking element, in which the connection between these components is a sealed connection.

FIGS. 10 and 11 respectively show a locking element and a metallic ring from an example of the device of the invention, in which the connection between these components is a mechanical connection.

FIG. 12 shows the components of FIGS. 10 and 11 and the way in which the mechanical connection between them takes place.

FIG. 13 shows an example of a device of the invention with a metallic ring and a locking element, where the connection between these components is a magnetic connection.

FIGS. 14 and 15 show a locking element of an example of the device of the invention to which a surgical lens can be adapted.

FIGS. 16 and 17 show the locking element of FIGS. 14 and 15 to which a surgical lens has been adapted.

### PREFERRED EMBODIMENT OF THE INVENTION

Some particular examples of devices according to the present invention are described below, making reference to the attached figures wherein the different parts that composed it can be observed.

### FIRST EXAMPLE

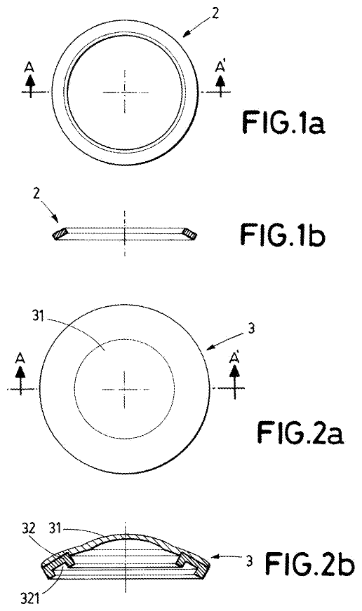

FIGS. 1a and 1b show a first example of a metallic ring (2) according to the invention which has a rectangular cross section. As can be seen, the rectangle constituting the cross section has a certain inclination, in such a way that the upper surface of the metallic ring (2) has a conical surface. This configuration is necessary for a good coupling with the coupling ring (32), as will be explained later. The dimensions of this metallic ring (2) can range from 11 mm to 24 mm.

FIGS. 2a and 2b show the locking element (3) which consists of the lens (31) and the coupling ring (32). The lens (3) has a convex shape similar to the lenses commonly used in this field. The coupling ring (32), on the other hand, has an essentially rectangular cross sectional shape with a cavity (321) on the lower side, which also has a rectangular shape which is complementary to the rectangular shape of the metallic ring (2) described above. In other words, the cavity (321) forms a rectangular-shaped recessed channel that runs along the entire circumference of the lower side of the coupling ring (32). As can be seen, the curvature of the lens (3) causes the channel formed by the cavity (321) of the coupling ring (32) to be oriented with a certain inward inclination. In addition, the coupling ring (32) has magnetic properties.

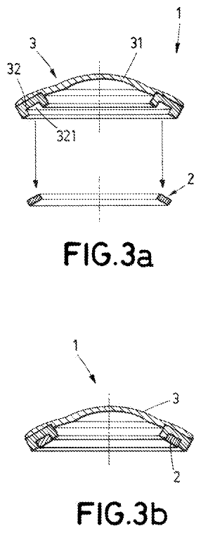

FIGS. 3a and 3b show the process of coupling the locking element (3) to the metallic ring (2). It can be observed that the metallic ring (2) fits perfectly into the channel formed on the bottom surface of the coupling ring (32). The magnetism of the coupling ring (32) attracts the metallic ring (2), thus preventing it from being unintentionally uncoupled.

### SECOND EXAMPLE

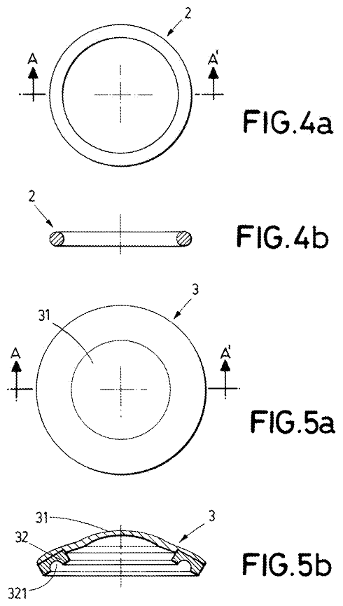

FIGS. 4a and 4b show a second example of a metallic ring (2) according to the invention which has a circular cross section. It would therefore be a ring (2) very similar to the well-known Flieringa ring.

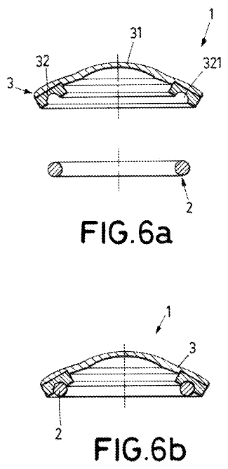

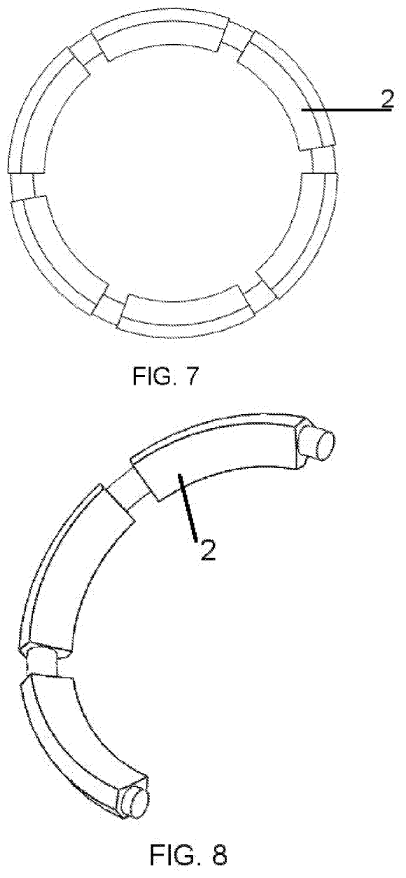

FIGS. 5a and 5b show a second example of the locking element (3) wherein the coupling ring (32) has a semicircular cross section cavity (321). Therefore, in this case, the lower surface of the coupling ring (32) has a semicircular channel that runs along its entire circumference. Also, in this example, the semicircular channel formed by the cavity (321) of the coupling ring (32) is oriented with a certain inward inclination, a consequence of the very curvature of the lens (31) to which it is fixed. As in the first example, the coupling ring (32) has magnetic properties. Finally, FIGS. 6a and 6b show the coupling of the locking element (3) on the metallic ring (2) of this second example of the device (1) according to the invention.

### THIRD EXAMPLE

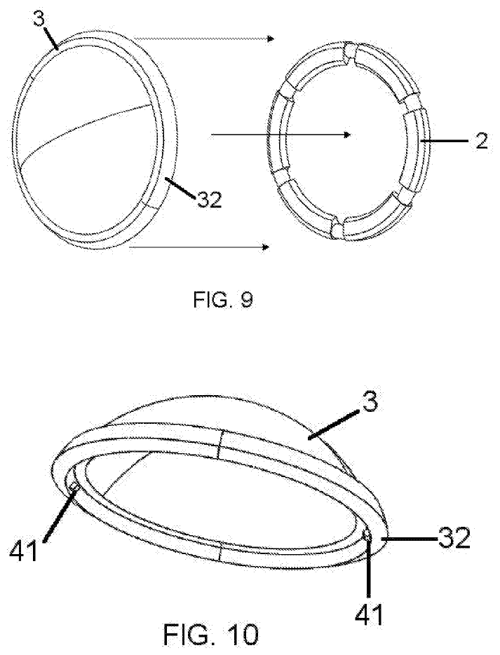

FIG. 9 shows a third example of a device of the invention, with a metallic ring (2) and a locking element (3), in which the connection between these components is a sealed connection.

The sealed connection consists of connection by means of a material of elastomeric nature of the coupling ring (32) with the metallic ring (2). For this purpose, the coupling ring (32) is made of an elastomer material, preferably medical silicone or polyurethane.

### FOURTH EXAMPLE

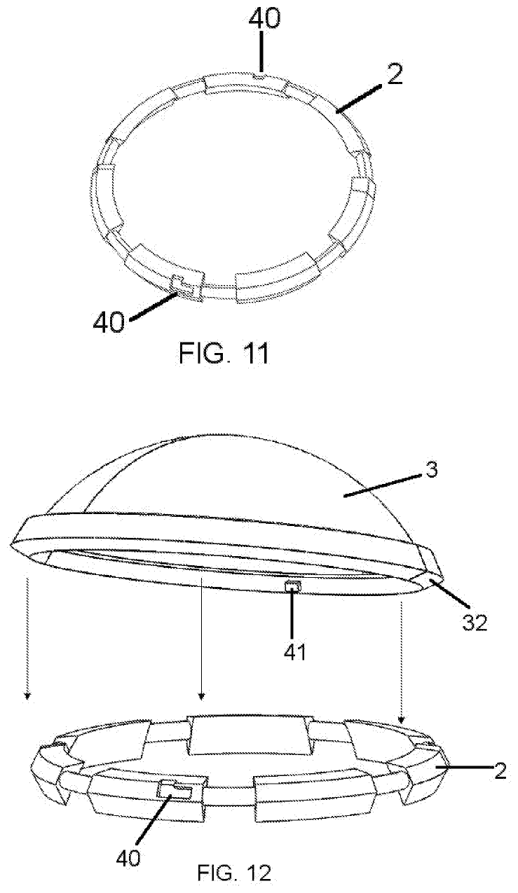

FIG. 12 shows an example of a device of the invention with a metallic ring (2) and a locking element (3), in which the joint between these components is a mechanical connection. In this example, the metallic ring (2) has grooves (40) and the coupling ring (32) has protrusions (41), in such a way that the metallic ring (2) can be fixed and/or sealed with the coupling ring (32).

### FIFTH EXAMPLE

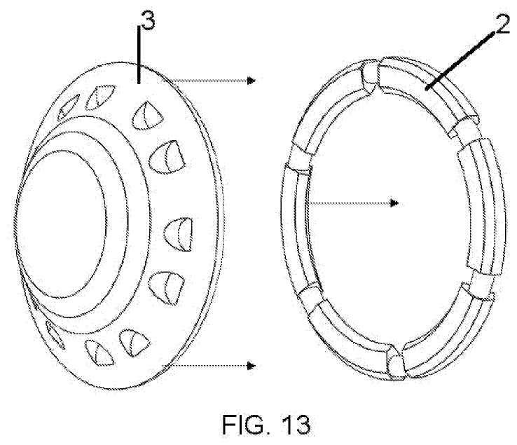

FIG. 13 shows an example of a device of the invention with a metallic ring (2) and a locking element (3), in which the connection between these components is a magnetic connection as described above.

### SIXTH EXAMPLE

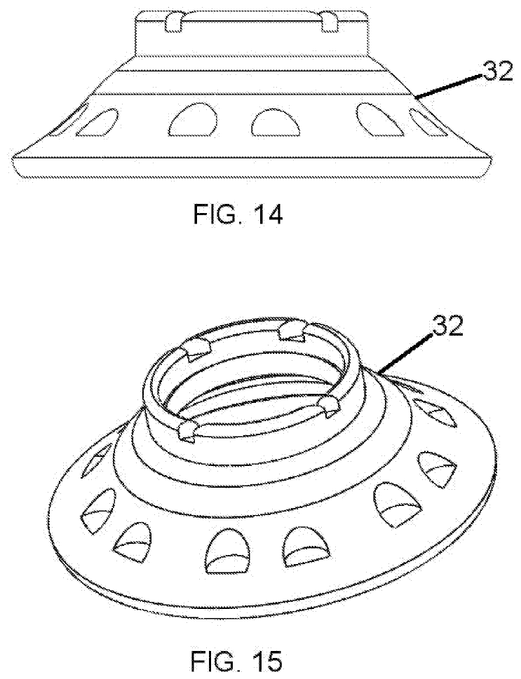

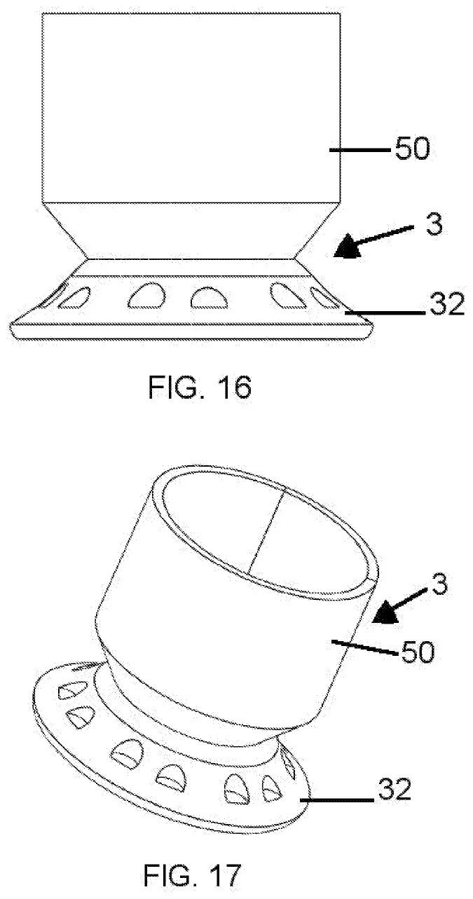

FIGS. 16 and 17 show an example of a coupling ring (32) of a locking element (3) of a device of the invention to which a surgical lens (50) has been adapted. FIGS. 14 and 15 show only the coupling ring (32) from FIGS. 16 and 17, which can be fitted with various types of surgical lenses.

1. A device for eye surgery, which comprises:
a metallic ring configured for suturing to the sclera of a patient's eye; and
a locking element which is couplable to the metallic ring, wherein the locking element comprises:
a lens, and
a coupling ring fixed to the lens along an entire circumference of an inner edge of the lens, the coupling ring being configured for coupling to the metallic ring.

2. The device according to claim 1, wherein the coupling ring has a cross section which has a cavity whose shape is complementary to a shape of a cross section of the metallic ring.

3. The device according to claim 2, wherein the cavity of the coupling ring is parallelepiped and the metallic ring has a correspondingly parallelepipedic cross section.

4. The device according to claim 2, wherein the cavity of the coupling ring is semicircular and the metallic ring has a circular cross section.

5. The device according to claim 1, wherein the coupling ring and the metallic ring include additional threads which provide for coupling the coupling ring and the metallic ring.

6. The device according to claim 1, further comprising fixing clips for the coupling between the coupling ring and the metallic ring.

7. The device according to claim 1, wherein the lens is a transparent plastic lens.

8. The device according to claim 8, wherein the lens is made of polymethylmethacrylate.

9. The device according to claim 1, wherein the lens is made of a material compatible with paracentesis.

10. The device according to claim 10, wherein the material compatible with paracentesis is silicone.

11. The device according to claim 1, wherein the lens is a lens for posterior segment surgery.

12. The device according to claim 1, wherein the coupling ring comprises a fixation system for placement of different types of lenses for posterior segment surgery and for combined penetrating and posterior segment keratoplasty surgery.

13. The device according to claim 1, wherein the connection between the coupling ring and the metallic ring is a magnetic connection, in which the coupling ring comprises a magnetic material and the metallic ring comprises a ferromagnetic material.

14. The device according to claim 1, wherein a connection between the coupling ring and the metallic ring is a magnetic connection, in which the coupling ring comprises a ferromagnetic material and the metallic ring comprises a magnetic material.

15. The device according to claim 13, wherein the magnetic material comprises neodymium magnets or electromagnets.

16. The device according to claim 1, wherein a connection between the coupling ring and the metallic ring is a mechanical connection in which the coupling ring has a mechanism of grooves and protrusions which facilitates fixing to the metallic ring.

17. The device according to claim 1, wherein a connection between the coupling ring and the metallic ring is a sealed connection, in which the coupling ring is made of an elastomer material.

18. The device according to claim 17, wherein the coupling ring is a medical silicone or polyurethane gasket.

19. The device according to claim 1, wherein the metallic ring is covered with one or more modules made of steel, titanium or polymer-based materials.

20. The device according to claim 1, wherein the metallic ring comprises at least one tab protruding from the metallic ring.

21. The device according to claim 1, wherein the coupling ring is made of steel, titanium, nitinol or polymer-based materials.
